(19) United States
(12) Patent Application Publication
(10) Pub. No.: US 2023/0301939 A1
(43) Pub. Date: Sep. 28, 2023

(54) NOVEL FORMULATIONS COMPRISING KETAMINE

(71) Applicant: Neurocentrx Pharma Ltd., Edinburgh (GB)

(72) Inventor: Carmel Reilly, Edinburgh (GB)

(21) Appl. No.: 17/253,828

(22) PCT Filed: Jun. 17, 2019
(86) PCT No.: PCT/GB2019/051684

§ 371 Date: Dec. 18, 2020

(30) Foreign Application Priority Data
Jun. 18, 2018 (GB) 1809976.2

(51) Int. Cl. A61K 31/135; A61K 9/48

(52) U.S. Cl. CPC A61K 31/135; A61K 9/4825; A61K 9/4858; A61K 9/4866

(57) ABSTRACT

Disclosed herein are oral, abuse resistant (tamper proof) formulations comprising ketamine. Each formulation is provided for oral use with a measured ketamine dose. These formulations are easier to track and trace and comprise abuse deterrent/tamper proof technology. In addition to having abuse deterrent properties, the formulations exhibit immediate or rapid API (ketamine and/or pharmaceutically acceptable salts, enantiomers, derivatives or metabolites thereof) release.

### RELATED APPLICATIONS

This application is a 35 U.S.C. § 371 national stage application of PCT Application No. PCT/GB2019/051684, filed on Jun. 17, 2019, which claims priority from United Kingdom Patent Application No. 1809976.2, filed on Jun. 18, 2018, the contents of each of which are incorporated herein by reference in their entireties. The above-referenced PCT International Application was published in the English language as International Publication No. WO 2019/243791 A1 on Dec. 26, 2019.

### FIELD OF THE INVENTION

The disclosure provides novel formulations comprising ketamine. In particular, the disclosure relates to abuse deterrent formulations for ketamine hydrochloride. The formulations may be for oral use in the treatment, prevention, management and/or control of a variety of clinical indications including pain, psychiatric/mental health/mood disorders and suicidal ideation and behaviour.

### BACKGROUND OF THE INVENTION

Ketamine is on the World Health Organisation's list of essential medicines for human health. Although it is best known medically as an anaesthetic for human and animal health, and is “approved” or “licensed” by regulatory authorities for this purpose, it is also used worldwide for pain relief, although not licensed for this use. Despite being discovered in the 1960's, Ketamine's full potential to treat a range of human diseases and disorders is only recently becoming understood.

Knowledge about ketamine's usefulness for certain patients is restricted to some specialists in each area, leaving many patients to suffer greatly, unable to work or participate fully in life and society. One licensed format is an injectable form, which means costly hospital stays, or appointments, and inconvenience for the patient.

There is an unmet medical and commercial need for oral ketamine delivery. Today Ketamine is approved for, and labelled as, an injectable anaesthetic, as a nasal spray and is used intramuscularly (IM) or intra-venously (IV). Two licensed human medical use forms are available, the racemic (R,S)-ketamine hydrochloride and the S-ketamine hydrochloride salt. It is used in this injectable form “off-label” to treat various pain conditions, mood disorders, psychiatric and mental health issues, and suicidal ideation and behaviour. It is not approved for any of these conditions.

Many studies with the IV form and patient experiences have led to a mushrooming of “ketamine-treatment-centres”, particularly in the US. In most treatment centres racemic ketamine hydrochloride is used.

Orally administered ketamine may have advantages to the patient, but it is not yet known if ketamine administered this way has commercially proven efficacy in any of the clinical areas aforementioned. As no oral form has been commercially available, almost all clinical studies have been academic-led with the commercially available IV form.

Ketamine is absorbable by intravenous, intramuscular, oral, and topical routes due to both its water and lipid solubilities. When administered orally, it undergoes first-pass metabolism, where it is biotransformed in the liver by CYP3A4 (major), CYP2B6 (minor), and CYP2C9 (minor) isoenzymes into norketamine (through N-demethylation) and finally dehydronorketamine. Intermediate in the biotransformation of norketamine into dehydronorketamine is the hydroxylation of norketamine into hydroxynorketamine by CYP2B6 and CYP2A6. Dehydronorketamine, followed by norketamine, is the most prevalent metabolite detected in urine. As the major metabolite of ketamine, norketamine is one-third to one-fifth as potent as an anesthetic, and plasma levels of this metabolite are three times higher than ketamine following oral administration. Bioavailability through the oral route reaches 17-20%; bioavailability through other routes are: 93% intramuscularly, 8-50% intranasally, 30% sublingually, and 30% rectally. Peak plasma concentrations are reached within a minute intravenously, 5 to 15 minutes intramuscularly, and 30 minutes orally. Ketamine's duration of action in a clinical setting is 30 minutes to 2 hours intramuscularly and 4 to 6 hours orally.

It is known that S-ketamine is more potent than the racemic mixture in humans and presents with more psychoactive side-effects. It is not known if R-ketamine provides efficacy with reduced side-effects in humans, though some data from small animals is available. A number of studies have suggested that S or esketamine has a more medically useful pharmacological action than R-ketamine or racemic ketamine. However, in mice it was found that the rapid antidepressant effect of R or arketamine was greater and lasted longer than that of esketamine. As such, as an antidepressant, the contrary has been stated (“R ketamine appears to be a potent and safe antidepressant relative to S ketamine”. It has also been stated that “R-ketamine is a longer-lasting antidepressant compared with rapastinel”.

The metabolites of ketamine seem to be important. Taken orally, ketamine that reaches the stomach is then subject to first-pass metabolism and largely converted to norketamine. Norketamine is postulated to provide pain relief and have similar clinical benefits to ketamine with reduced side-effects. It is not known if norketamine is an effective clinical treatment at this time. Other metabolites are postulated to be important, particularly those metabolites that are derived from R-ketamine, namely 6-hydroxynorketamine or R-hyroxynorketamine (HNK) and specifically (2S,6S;2R,6R)-HNK, is responsible for antidepressant-like effects of ketamine in mice. Specifically, administration of (2R,6R)-HNK demonstrated ketamine-type antidepressant-like effects, and preventing the metabolic conversion of ketamine into HNK blocked the antidepressant-like effects of the parent compound, but there is no evidence to support a human benefit of (2R,6R)-HNK at this time.

Optical Isomers of Ketamine:

Ketamine exists in different optical isomers and salt forms. Some of these forms and metabolites have been shown to have positive clinical utility, with increased or decreased side-effects. For many isomers, enantiomers and salt forms, the evidence for human utility is lacking, apart from the S-enantiomer, and the racemic R,S hydrochloride salt which are available as injectable licensed products.

The optical rotation of a given enantiomer of ketamine can vary between its salts and free base form. The free base form of (S) ketamine exhibits dextrorotation and is therefore labelled (S) (+) ketamine. However, its hydrochloride salt shows levorotation and is thus labelled (S) (−) ketamine hydrochloride. The difference originates from the conformation of the cyclohexanone ring. In both the free base and the hydrochloride, the cyclohexanone ring adopts a chair conformation, but the orientation of the substituents varies. In the free base, the o-chlorophenyl group adopts an equatorial position and the methylamino group adopts an axial position. In the hydrochloride salt, the positions are reversed, with the o-chlorophenyl group axial and the methylamino group equatorial. Not all salts of ketamine show different optical rotation to the free base: (S)-ketamine (R,R)-tartrate is levorotatory, like (S) ketamine.

Ketamine Mechanism:

The full mechanism, pathways and combination of pathway effects required for ketamine to be effective in different clinical areas is not currently understood. Ketamine is known mostly as an NMDA antagonist, and more recently AMPAR. However ketamine also has effects at the opioid, sigma, dopamine, serotonin, muscarinic, nicotinic, and oestrogen receptors and inhibits cholinesterase, serotonin, norepinephrine and dopamine reuptake inhibitor, PCP site 2 ligand, blocks sodium and calcium channels, HCN1 cation channels and inhibits nitric oxide synthase.

With a few exceptions (including interactions with the D2high receptor, nicotinic acetylcholine receptors (by metabolites), ERα, and HCN1 channels), these actions are far weaker than ketamine's antagonism of the NMDA receptor, though it has been postulated that allosteric binding at other sites may augment ketamine's activity at these sites.

Metabolites of ketamine including dehydronorketamine, hydroxynorketamine, and norketamine have been found to act as negative allosteric modulators of the a7 nicotinic acetylcholine receptor in the KXa7R1 cell line (HEK293 cells transfected with rat nicotinic acetylcholine receptor genes) with subanaesthetic and nanomolar potencies (e.g., IC50=55 nM for dehydronorketamine), whereas ketamine itself was inactive at the same concentrations (<1 μM). These findings suggest that metabolites may contribute importantly to the pharmacodynamics of ketamine by means other than NMDA receptor antagonism.

It has yet to be fully understood how ketamine mediates its robust and rapid-onset antidepressant effects. In any case, it has been elucidated that acute blockade of NMDA receptors in the brain results in an activation of α-amino-3-hydroxy-5-methyl-4-isoxazolepropionic acid receptors (AMPA receptors), which in turn modulate a variety of downstream signalling pathways to influence neurotransmission in the limbic system and mediate antidepressant effects of NMDA receptor antagonists like ketamine. Such downstream actions of this activation of AMPA receptors include upregulation of brain-derived neurotrophic factor (BDNF) and activation of its signalling receptor tropomyosin receptor kinase B (TrkB), activation of the mammalian target of rapamycin (mTOR) pathway, deactivation of glycogen synthase kinase 3 (GSK-3), and inhibition of the phosphorylation of the eukaryotic elongation factor 2 (eEF2) kinase. In addition to blockade of the NMDA receptor, an active metabolite of ketamine known as hydroxynorketamine, which does not interact importantly with the NMDA receptor but nonetheless indirectly activates AMPA receptors similarly, may also or alternatively be involved in the rapid-onset antidepressant effects of ketamine. Recent research has elucidated that an acute inhibition or sort of “reset” of the lateral habenula, a part of the brain in the limbic system that has been referred to as the “anti-reward centre” (projecting to and inhibiting the mesolimbic reward pathway and modulating other limbic areas), may be responsible for the antidepressant effects of ketamine.

The disclosure provides oral ketamine formulations which are encapsulated, exhibit a significant degree of tamper/abuse deterrence, exhibit acceptable (or suitable) dissolution profile, are stable in storage.

### SUMMARY OF THE INVENTION

Disclosed herein are oral, abuse resistant (tamper proof) formulations comprising ketamine. Each formulation is provided for oral use with a measured ketamine (aka the “active pharmaceutical ingredient (API)) dose. These formulations are easier to track and trace and comprise abuse deterrent/tamper proof technology. In addition to having abuse deterrent properties, the formulations exhibit an appropriate API release profile. For example, the formulations may exhibit a controlled and/or immediate or rapid active (ketamine) release.

The disclosure provides novel formulations comprising ketamine and/or pharmaceutically acceptable salts, enantiomers, derivatives or metabolites thereof.

The disclosed formulations exhibit abuse deterrence, active (ketamine) stability and favourable dissolution profiles.

The disclosed formulations may be for oral administration.

The disclosed formulations may not be prepared in tablet form.

The disclosed formulations may be provided as capsules. For example, the disclosed formulations may be encapsulated in a hard capsule.

The disclosed formulations may be resistant to misuse and abuse.

The disclosed composition may exhibit a predetermined API release profile. For example, the formulations may exhibit controlled release characteristics. For example, the API may be released (form the formulation) slowly or rapidly.

It should be noted that throughout this specification the term “comprising” is used to denote that embodiments of the invention “comprise” the noted features and as such, may also include other features. However, in the context of this invention, the term “comprising” may also encompass embodiments in which the invention “consists essentially of” the relevant features or “consists of” the relevant features.

In a first aspect, the disclosure provides novel formulations, which formulations may comprise ketamine.

The disclosed formulations may comprise a ketamine suspension.

Ketamine is a water-soluble phencyclidine derivative with an asymmetric carbon atom; it has two enantiomers. Ketamine is metabolized in the liver via N-demethylation and ring hydroxylation pathways and the main metabolites are norketamine and its hydroxylated derivatives.

The formulations may comprise a pharmaceutically acceptable salt of ketamine.

The formulations may comprise any form of ketamine which is a NMDA antagonist and/or modulator of the AMPAR (α-amino-3-hydroxy-5-methyl-4-isoxazolepropionic acid receptor).

The formulations may comprise ketamine enantiomers, derivatives and/or metabolites. The formulations described herein (which formulations are for oral administration) may be metabolised by the body to one or more ketamine metabolites.

The formulations may comprise ketamine hydrochloride (a non-competitive NMDA receptor antagonist).

The formulations may comprise racemic (R,S) ketamine hydrochloride.

By way of example, the formulations described herein may comprise one or both of the R-/S-Ketamine stereoisomers. For example, the formulation may comprise either R-ketamine or S-Ketamine.

The term “ketamine metabolites” may comprise the following compounds:

2R,6R-Hydroxynorketamine hydrochloride (this enhances AMPA currents; decreases D-serine (a NMDA co-agonist) and lacks ketamine-related side effects).

2S,6S-Hydroxynorketamine hydrochloride (this decreases D-serine (a NMDA co-agonist); it is an antidepressant).

cis-6-Hydroxynorketamine hydrochloride (this enhances AMPA currents: it is an antidepressant)

(S)-(+)-Ketamine hydrochloride (a NMDA receptor antagonist; enantiomer of ketamine hydrochloride with neuroprotective effects).

Norketamine hydrochloride (a potent, non-competitive NMDA antagonist; antinociceptive) (R)-Norketamine hydrochloride (a NMDA receptor modulator and an analgesic)

(S)-Norketamine hydrochloride (a NMDA receptor modulator and an analgesic).

It should be noted, that the term “ketamine” or “active” as used hereinafter, shall be taken to refer to and embrace all of the ketamine types, salts, derivatives, enantiomers and/or metabolites described above.

The formulation may comprise any suitable amount of ketamine. For example, of the total volume of a formulation provided by this invention, the formulation may comprise 1%-70% (v/v %) ketamine hydrochloride. For example, the formulation may comprise 2%, 3%, 4%, 5%, 6%, 7%, 8%, 9%, 10%, 15%, 20%, 25%, 30%, 35%, 40%, 45%, 50%, 55%, 60% or 65% (v/v %) ketamine hydrochloride.

For example, a formulation of this disclosure may comprise about (for example +/−1, 2, 3, 4 or 5% (v/v %)) 8% (v/v %) ketamine hydrochloride.

The formulation further comprises one or more excipients, diluents and/or carriers.

The excipients, diluents and/or carriers may be selected on the basis of function. For example the formulations described herein may comprise excipients which are:
(i) diluents;
(ii) carriers.
(iii) solvents
(iv) plasticisers
(v) surfactants
(vi) lubricant
(vii) dispersants
(viii) viscosity modifiers
(ix) bioavailability modifiers
(x) emulsifiers
(xi) penetration enhancers
(xii) release modifiers
(xiii) gelling agents
(xiv) stabilisers
(xv) antioxidants
(xvi) pH regulators/acidity modifiers; and
(xvii) thermal stability/setting regulators.

The precise amount (for example volume (v/v %)) of each excipient, diluent and/or carrier may vary depending on the desired properties of the formulation. For example, the inventors have found that by adjusting the relative amounts of any carrier compounds and any viscosity modifiers present in the formulation, it is possible to modulate (for example increase or decrease) the rate of release of the active agent. In some embodiments the amount of carrier agent used in the formulation may be lower than the amount of viscosity modifier used in the formulation. In other embodiments, the amount of carrier agent used in the formulation may be higher than the amount of viscosity modifier used in the formulation. One of skill will appreciate that useful formulations must include an active pharmaceutic ingredient (API: in this case ketamine as described herein) in an amount sufficient to achieve a target dose while at the same time maintaining the level of any viscosity modifier to ensure abuse deterrence and a sufficient concentration of carrier excipient(s) to ensure the formulation can be successfully processed. The formulations described herein meet these criteria.

In one embodiment, a formulation according to the present disclosure may comprise an amount of ketamine together with one or more excipients. The excipients may comprise, for example, various carrier compounds/compositions, viscosity modifier(s) and, optionally, an antioxidant.

Suitable carriers may include, for example co-polymers (including those selected from the group consisting of triglycerides, poloxamers, polysorbates, polyethoxylates, polyethylene glycols and poly-glycerides). Suitable carriers may also include, for example, poloxamer 124, polysorbate 80, polysorbate 20, kolliphor EL, Kolliphor RH40, Polyethylene glycol 1000, 1500, 2000, 6000 and 8000, Kolliphor HS15, Gelucire 44/14, Gelucire 48/16, Gelucire 50/13. A carrier suitable for use in a formulation of this invention may comprise, for example polyethylene glycol (PEG). For example, a useful carrier may comprise low molecular weight PEG, for example PEG 1500 or PEG 6000.

Where the formulation comprises PEG 1500, the formulation may comprise anywhere between about 1% and 50% (v/v %) PEG 1500. For example, a useful formulation may comprise about 7%, 8%, 9%, 10%, 15%, 20%, 25%, 26%, 27%, 30%, 40%, 41%, 42%, 43%, 44%, 45%, 46%, 47%, 48% or 49% (all “%” values being v/v %) PEG 1500. For example, a formulation for use may comprise 9.19% (v/v %), 27.57% (v/v %) or 45.95% (v/v %) PEG 1500.

The formulation may comprises PEG 6000; for example, the formulation may comprise anywhere between about 1% and 20% (v/v %) PEG 6000. For example, a useful formulation may comprise about 2%, 3%, 4%, 5%, 6%, 7%, 8%, 9%, 10%, 12%, 15%, 17% or 19% (all “%” values being v/v %) PEG 6000. For example, a formulation for use may comprise 4.6% (v/v %) or 9.19% (v/v %) PEG 6000.

Formulation testing revealed that a quantity of PEG in the formulation may assist in rendering the formulation resistant to tamper and/or abuse.

One of skill will appreciate that the actual amount of PEG 1500 and/or 6000 used will depend on (i) the desired properties of the formulation (e.g. desired viscosity, stability, dissolution (of the active ingredient: ketamine) and/or level of abuse deterrence) and (ii) the amount (v/v %) of the active and/or any other excipients/carriers and/or diluents used.

Other (or additional) carrier(s) for use may comprise Gelucire 44/14, Polysorbate 80 and/or Gelucire 48/16.

Gelucire 44/14 may be used at an amount equivalent between about 5% and 70% (v/v %). For example, a useful formulation may comprise about 6%, 7%, 8%, 9%, 10%, 15%, 19%, 20%, 21%, 30%, 35%, 40%, 45%, 49%, 50%, 51%, 52%, 53%, 54%, 55%, 56%, 57%, 58%, 59%, 60%, 65% or 70% (all “%” values being v/v %) Gelucire 44/14. For example, a formulation for use may comprise 20.68% (v/v %), 50.55% (v/v %), 55.14% (v/v %) or 59.74% (v/v %) Gelucire 44/14. One of skill will appreciate that the actual amount of Gelucire 44/14 used will depend on (i) the desired properties of the formulation (viscosity, stability, dissolution of the active ingredient (ketamine) and/the level of abuse deterrence) and (ii) the amount (v/v %) of the active and/or any other excipients/carriers and/or diluents used.

Polysorbate 80 may be used at an amount equivalent between about 1% and 30% (v/v %). For example, a useful formulation may comprise about 2%, 3%, 4%, 5%, 6%, 7%, 8%, 9%, 10%, 11%, 12%, 13%, 14%, 15%, 17%, 20%, 22%, 25%, 27% or 29% (all “%” values being v/v %) Polysorbate 80. For example, a formulation for use may comprise 4.6% (v/v %), 11.49% (v/v %) or 13.79% (v/v %) Polysorbate 80. One of skill will appreciate that the actual amount of Polysorbate 80 used will depend on (i) the desired properties of the formulation (viscosity, stability, dissolution of the active ingredient (ketamine) and/the level of abuse deterrence) and (ii) the amount (v/v %) of the active and/or any other excipients/carriers and/or diluents used.

Gelucire 48/16 may be used at an amount equivalent between about 10% and 70% (v/v %). For example, a useful formulation may comprise about 11%, 15%, 20%, 25%, 30%, 35%, 40%, 45%, 49%, 50%, 51%, 52%, 55%, 60%, 65% or 69% (all “%” values being v/v %) Gelucire 48/16. For example, a formulation for use may comprise 50.55% (v/v %) Gelucire 48/16. One of skill will appreciate that the actual amount of Gelucire 48/16 used will depend on (i) the desired properties of the formulation (viscosity, stability, dissolution of the active ingredient (ketamine) and/the level of abuse deterrence) and (ii) the amount (v/v %) of the active and/or any other excipients/carriers and/or diluents used.

As stated, a formulation of this disclosure may comprise a viscosity modifier. The viscosity modifier may comprise a gum, for example a gellan gum having two acyl substituents-acetate and glycerates (with an average content of one glycated per repeat and acetate per every two repeats).

A useful gum may be a high acyl gellan gum having two acyl substituents and perhaps a typical weight average molecular weight of at least 5×10⁵, 7×10⁵ Daltons, 1×10⁶ Daltons or preferably between about 1-2×10⁶ Daltons (one of skill will appreciate that the molecular weight of a gellan gum can be determined by any suitable method including the methods disclosed in U.S. Pat. No. 6,242,035 the contents of which are incorporated herein by reference). A suitable gum may comprise a repeating unit of the tetrasaccharide polymer which consists of two residues of D-glucose and one of each residues of L-rhamnose and D-glucuronic acid. The tetrasaccharide repeat may have the following structure: [D-Glc(β1→4)D-GlcA(β1→4)D-Glc(β1→4)L-Rha(α1→3)]ₙ. For example, the gum (and thus a formulation according to this invention) may comprise Kelcogel® CGHA™ or Kelcogel® LT100 (from CP kelco). A useful gellan gum may have a set temperature of 30-50° C.

Kelcogel CGHA may be used at an amount equivalent between about 20% and 50% (v/v %) or between about 25% and 40% (v/v %). For example, a useful formulation may comprise about 21%, 25%, 30%, 35%, 40%, 45%, 40%, 45%, 49%, 50%, 51%, 52%, 55%, 60%, 65% or 69% (all “%” values being v/v %) Kelcogel CGHA. For example, a formulation for use may comprise 27.57% (v/v %) or 32.17% (v/v %) Kelcogel CGHA. One of skill will appreciate that the actual amount of Kelcogel CGHA used will depend on (i) the desired properties of the formulation (viscosity, stability, dissolution of the active ingredient (ketamine) and/the level of abuse deterrence) and (ii) the amount (v/v %) of the active and/or any other excipients/carriers and/or diluents used.

When combined with certain excipients, ketamine (or the API) is prone to some level of degradation. Degradation in the presence of selected excipients (for example PEG1500, PEG6000, Gelucire 44/14, Poloxamer 124, kolliphor RH40, Kolliphor EL, Polysorbate 20, Polysorbate 80, Miglyol 812N) was observed after a period of storage (for example 4 weeks) at a particular temperature/humidity (40° C./75% RH). The inventors observed that in the presence of certain excipients, the API (ketamine) was prone to oxidation. Accordingly, the formulations disclosed herein may comprise an antioxidant. A suitable antioxidant may comprise, for example, Butylated hydroxytoluene (BHT). A formulation comprising Butylated hydroxytoluene (BHT) may use, for example about 0.01% to about 20% (v/v %). For example, a useful formulation may comprise about 0.05%, 0.1%, 0.2%, 0.5%, 1%, 2%, 3%, 4%, 5%, 6%, 7%, 8%, 9%, 10%, 11%, 12%, 13%, 14%, 15%, 16%, 17%, 18%, or 19% (all “%” values being v/v %) Butylated hydroxytoluene (BHT). For example, a formulation for use may comprise 0.1% (v/v %) Butylated hydroxytoluene (BHT). One of skill will appreciate that the actual amount of Butylated hydroxytoluene (BHT) used will depend on (i) the desired properties of the formulation (viscosity, stability, dissolution of the active ingredient (ketamine) and/the level of abuse deterrence) and (ii) the amount (v/v %) of the active and/or any other excipients/carriers and/or diluents used. In all cases, an antioxidant amount of BHT should be used.

The formulations described herein may be contained within a capsule shell. For example, a formulation of this disclosure may be contained within capsules comprising gelatin or hypromellose (HPMC).

A formulation according to this disclosure may comprise ketamine hydrochloride together with one or more excipients, carriers or diluents, selected from the group consisting of:
(i) PEG 1500;
(ii) PEG6000;
(iii) Gelucire 44/14;
(iv) Polysorbate 80;
(v) Gelucire 48/16;
(vi) Kelcogel CGHA; and
(vii) Butylated hydroxytoluene (BHT)

A suitable formulation may comprise ketamine hydrochloride and
(i) Kelcogel CGHA;
(ii) Gelucire 44/14; and
(iii) Butylated hydroxytoluene (BHT)

A suitable formulation may comprise ketamine hydrochloride and
(i) Kelcogel CGHA;
(ii) PEG 6000
(iii) Gelucire 44/14
(iv) Polysorbate 80; and
(v) Butylated hydroxytoluene (BHT)

A suitable formulation may comprise ketamine hydrochloride and
(i) Kelcogel CGHA;
(ii) PEG 6000
(iii) Gelucire 44/14; and
(iv) Butylated hydroxytoluene (BHT)

A suitable formulation may comprise ketamine hydrochloride and
(i) Kelcogel CGHA;
(ii) PEG 1500;
(ii) Polysorbate 80; and
(iv) Butylated hydroxytoluene (BHT)

A suitable formulation may comprise ketamine hydrochloride and
(i) Kelcogel CGHA;
(ii) PEG1500;
(iii) Gelucire 48/16; and
(iv) Butylated hydroxytoluene (BHT)

A suitable formulation may comprise ketamine hydrochloride and
(i) Kelcogel CGHA;
(ii) PEG1500;
(iii) Gelucire 44/14;
(iv) Polysorbate 80; and
(vi) Butylated hydroxytoluene (BHT)

A suitable formulation may comprise ketamine hydrochloride and
(i) Kelcogel CGHA
(ii) Gelucire 44/14
(iii) BHT

A suitable formulation may comprise ketamine hydrochloride and
(i) Kelcogel CGHA
(ii) Gelucire 44/14
(iii) Polysorbate 80
(iv) BHT

A suitable formulation may comprise ketamine hydrochloride and
(i) Kelcogel CGHA
(ii) PEG1500
(iii) Gelucire 44/14
(iv) BHT

A suitable formulation may comprise ketamine hydrochloride and
(i) Kelcogel CGHA
(ii) PEG1500
(iii) Gelucire 44/14
(iv) Polysorbate 80
(v) BHT

A suitable formulation may comprise ketamine hydrochloride and
(i) Kelcogel CGHA
(ii) PEG1500
(iii) Gelucire 44/14
(iv) BHT

A suitable formulation may comprise ketamine hydrochloride and
(i) Kelcogel CGHA
(ii) PEG1500
(iii) Gelucire 44/14
(iv) Polysorbate 80
(v) BHT

A series of exemplary (but not limiting) formulations are detailed in the tables below. Each table details six different ketamine hydrochloride based formulations. In each case, the formulation is specified in terms of the amount (v/v %) of each component.

TABLE 1
Example formulations comprising ketamine hydrochloride
Formulation Compositions %

| | Formulations - Iteration 1 | | | | | |
|---|---|---|---|---|---|---|
| Material | 1.1 | 2.1 | 3.1 | 4.1 | 5.1 | 6.1 |
| Kelcogel CGHA | 32.17 | 27.57 | 32.17 | 32.17 | 32.17 | 32.17 |
| PEG1500 | — | — | — | 45.95 | 9.19 | 27.57 |
| PEG6000 | — | 4.60 | 9.19 | — | — | — |
| Gelucire 44/14 | 59.74 | 55.14 | 50.55 | — | — | 20.68 |
| Polysorbate 80 | — | 4.60 | — | 13.79 | — | 11.49 |
| Gelucire 48/16 | — | — | — | — | 50.55 | — |
| BHT | 0.10 | 0.10 | 0.10 | 0.10 | 0.10 | 0.10 |
| Ketamine HCl | 8.00 | 8.00 | 8.00 | 8.00 | 8.00 | 8.00 |
| Total | 100.00 | 100.00 | 100.00 | 100.00 | 100.00 | 100.00 |

TABLE 2
Example formulations comprising ketamine hydrochloride
Formulation Compositions %

| | Formulations - Iteration 2 | | | | | |
|---|---|---|---|---|---|---|
| Material | 1.2 | 7.1 | 8.1 | 4.2 | 9.1 | 6.2 |
| Kelcogel CGHA | 36.76 | 36.76 | 32.16 | 32.16 | 32.16 | 32.16 |
| PEG1500 | — | — | 29.87 | 36.76 | 9.19 | 29.87 |
| Gelucire 44/14 | 55.14 | 48.25 | 29.87 | 10.34 | 50.55 | 22.98 |
| Polysorbate 80 | — | 6.89 | — | 12.64 | — | 6.89 |
| BHT | 0.10 | 0.10 | 0.10 | 0.10 | 0.10 | 0.10 |
| Ketamine HCl | 8.00 | 8.00 | 8.00 | 8.00 | 8.00 | 8.00 |
| Total | 100.00 | 100.00 | 100.00 | 100.00 | 100.00 | 100.00 |

As stated, the formulations disclosed herein are designed to deter abuse. In other words, the disclosed formulations comprise (or exhibit) abuse deterrent properties.

The term “abuse” may embrace any intentional, non-therapeutic use of a drug product or substance, to achieve a desirable psychological or physiological effect. By way of example, a subject intent on abusing a drug product, for example a prescribed drug product, may crush or dissolve said drug product and subsequently attempt to snort, smoke or inject the crushed or dissolved drug product. For example, within the field of opioid-based drugs, abuse is common, with those addicted to opioids attempting to extract the opioid component from the opioid based drug in a form that can be, for example, injected, snorted or smoked.

The formulations described herein comprise, one or more technologies designed to deter abuse.

For example, the formulations comprise chemical barriers that limit drug release from the formulation and/or render the formulation difficult (or impossible) to process to a form that can be injected or snorted. For example, the chemical barriers (viscosity modifiers and the like) can limit release of the active agent (in this case ketamine) even after processing (crushing, sieving, heating and the like).

The effectiveness or efficacy of any particular formulation depends on the active agent dissolving in, for example, the fluids of the gastrointestinal tract. The rate of dissolution is important as this determines how much of the active agent is released and is available for use in the treatment and/or prevention of a diseases and/or disorder. The formulations described herein may be immediate release formulations. In the present case, the dissolution of the various formulations was tested and the inventors determined that all formulations achieved the required level of dissolution. Suitable dissolution rates may require that a certain percentage of the active agent (in this case ketamine hydrochloride) is recovered after some predetermined time. For example, anywhere between about 1-100% of the active agent may be recovered in dissolution testing. For example, about 10%, about 20%, about 30%, about 40%, about 50%, about 60%, about 70%, about 80% about 90% or about 95% of the active agent (ketamine hydrochloride) may be recovered under dissolution testing. It should be noted that within the context of dissolution testing, the term “x %” means a percentage of the total about of active agent loaded into the formulation. For example any given formulation may be thought of as containing (before dissolution testing) an amount equivalent to 100% of the active agent. Under dissolution testing conditions the amount of active agent released is determined and this amount is expressed as a percentage of the total amount of the active agent originally provided within the formulation.

As stated, the dissolution profile of any given formulation may be assessed over a predetermined period of time; for example from about 1 minute to about 60 minutes. The dissolution profile may be assessed over, for example, about 10 minutes, about 15 minutes, about 20 minutes, about 25 minutes, about 30 minutes, about 35 minutes, about 40 minutes, about 45 minutes, about 50 minutes or about 55 minutes.

An acceptable dissolution profile may be, for example, 80% recovery (of the active agent (ketamine hydrochloride) after 30-45 minutes.

Thus the formulations described herein may be formulations for oral administration.

The formulations described herein have a PK and pharmacokinetic profile which differs markedly from other (prior art) ketamine formulations. Formulations comprising ketamine that are formulated for nasal, IV or IM administration, tend to be absorbed into the bloodstream and avoid early first pass metabolism. When administered orally, the bioavailability of ketamine is much lower and is subject to first pass metabolism into a number of different metabolites. Further, the formulation avoids the metallic taste which is characteristic of some formulations. Ketamine metabolites are postulated to be important in eliciting ketamine's pain relieving and anti-depressant and anti-suicidal effects.

The formulations provided by this disclosure may have a variety of clinical applications. As such, an aspect of this disclosure provides one or more of the formulations described herein, for use in medicine or for use as a medicament.

For example, the various formulations may be applied (i.e. may be for use in) the treatment or prevention of one or more of the following indications.

The formulations described herein may be for use in the management, treatment and/or prevention of pain. Further, one or more of the disclosed formulations may be used in the treatment and/or prevention (or control) of suicidal ideation and behaviour associated with pain.

It should be noted that the term “pain” embraces any pain associated with mental health, neurological, dermatological disorders, psychiatric disorders, wounds, minor surgical procedures, pen-operative pain; inflammatory disorders, cancer, asthma, chronic obstructive airway disease, painful procedures (for example in emergency departments) and emergency surgery in field conditions in trauma situations or war zones. The formulations described herein may also be for use in the management, treatment or prevention of post-surgery pain; opioid use; hyperalgesia; neuropathic pain; ischemic limb pain; peripheral neuropathies; complex regional pain syndrome type I and type II; phantom limb pain; post-herpetic neuralgia; Chronic ischemic monomelic neuropathy from critical limb ischemia; peripheral ischemic neuropathies; ischemic neuropathies associated with diabetes; complex pain across indications that are both inflammatory and neuropathic; clinical features across indications that include hyperalgesia, allodynia, central sensitisation, wind-up pain, dysaesthesias, paraesthesia, paroxysmal pain, neuropathic pain and tolerance (to other medication), and pain inadequately controlled by existing drug treatments.

The formulations of this disclosure may also be for use as pain relief in patients who require elevated blood pressure, maintenance of airway reflexes/skeletal muscle tone, or who may benefit from Ketamine's bronchodilatory properties.

A further aspect provides a method of treating pain (or suicidal ideation and/or behaviour associated therewith), said method comprising administering a subject in need thereof (for example a subject suffering from or predisposed to, any of the pain indications described above) ketamine in the form of a formulation described herein.

One or more of the formulations described herein may also be for use in the treatment, prevention, control and/or management of certain psychiatric disorders. Psychiatric disorders that may be treated by any of the formulations described herein may include, for example, bipolar disorder, cyclothymia, disruptive mood dysregulation disorder, dysthymia, major depressive disorder, premenstrual dysphoric disorder, seasonal affective disorder (SAD), mania, hypomania, treatment resistant depression, post-partum depression, atypical depression (AD), melancholic depression, psychotic major depression, catatonic depression, double depression, depressive personality disorder, depressive disorder not otherwise specified; recurrent brief depression; minor depressive disorder; bipolar disorder—bipolar I, bipolar II, cyclothymic bipolar disorder; substance-induced mood disorders, addiction and withdrawal (e.g.: alcohol, benzodiazepines); mood disorder secondary to another medical condition (e.g.: dementias, metabolic disorders, endocrine disorders, thyroid abnormalities, cardiovascular diseases (heart attack), pulmonary disease (chronic obstructive pulmonary disease), cancer and autoimmune diseases.

Again, the various formulations described herein may each be used in the treatment and/or prevention (or control) of suicidal ideation and behaviour associated with any of the abovementioned psychiatric disorders.

A further aspect provides a method of treating a psychiatric disorder (including any of the psychiatric disorders described above: (or suicidal ideation and/or behaviour associated therewith), said method comprising administering a subject in need thereof (for example a subject suffering from or predisposed to, any of the psychiatric disorders described above) ketamine in the form of a formulation described herein.

One or more of the formulations described herein may also be for use in the treatment, prevention, control and/or management of certain mental health disorders. Mental health disorders that may be treated by any of the formulations described herein may include, for example, post-traumatic stress disorders, severe and acute anxiety, eating disorders; neurodevelopmental disorders, personality disorders, psychotic disorders, substance use disorders.

The various formulations described herein may each be used in the treatment and/or prevention (or control) of suicidal ideation and behaviour associated with any of the abovementioned mental health disorders.

A further aspect provides a method of treating a mental health disorder (including any of the psychiatric disorders described above: (or suicidal ideation and/or behaviour associated therewith), said method comprising administering a subject in need thereof (for example a subject suffering from or predisposed to, any of the mental health disorders described above) ketamine in the form of a formulation described herein.

One or more of the formulations described herein may also be for use in the treatment, prevention, control and/or management of certain neurological disorders. Neurological disorders that may be treated by any of the formulations described herein may include, for example, various forms of palsy, palsy/writing and inflammation associated with Parkinson's disease, the side-effects of Parkinson's disease drugs, multiple sclerosis, Parkinson's plus diseases, migraine, Alzheimer's disease, epilepsy, L-dopa-Induced Dyskinesias and movement disorders.

The various formulations described herein may each be used in the treatment and/or prevention (or control) of suicidal ideation and behaviour associated with any of the abovementioned neurological disorders.

A further aspect provides a method of treating a neurological disorder (including any of the neurological disorders described above: (or suicidal ideation and/or behaviour associated therewith)), said method comprising administering a subject in need thereof (for example a subject suffering from or predisposed to, any of the neurological disorders described above) ketamine in the form of a formulation described herein.

One or more of the formulations described herein may also be for use in the treatment, prevention, control and/or management of certain dermatological disorders. Dermatological disorders that may be treated by any of the formulations described herein may include, for example, wounds; trigeminal nerve root injuries, truncal diabetic radiculitis, Dermatological and immunological conditions due to nerve lesions carpal tunnel syndrome, herpes zoster, neuropeptides, trigeminal trophic syndrome, herpes zoster infection, carpal tunnel syndrome, trigeminal trophic syndrome, sarcoidosis, vasculitic syndromes, mononeuritis or asymmetric sensorimotor neuropathy, polyarteritis nodosa, Churg-Strauss syndrome, Wegener granulomatosis, and cryoglobulinemia.

The various formulations described herein may each be used in the treatment and/or prevention (or control) of suicidal ideation and behaviour associated with any of the abovementioned dermatological health disorders.

A further aspect provides a method of treating a dermatological disorder (including any of the dermatological disorders described above: (or suicidal ideation and/or behaviour associated therewith)), said method comprising administering a subject in need thereof (for example a subject suffering from or predisposed to, any of the dermatological disorders described above) ketamine in the form of a formulation described herein.

One or more of the formulations described herein may also be for use in the treatment, prevention, control and/or management of erythromelalgia, a rare inherited clinical syndrome of intermittently red, hot, painful extremities. This syndrome usually affects the lower extremities (predominantly the feet) but may also involve the upper extremities (predominantly hands) and rarely concomitantly involves the face.

The various formulations described herein may each be used in the treatment and/or prevention (or control) of suicidal ideation and behaviour associated with erythromelalgia.

A further aspect provides a method of treating erythromelalgia (or suicidal ideation and/or behaviour associated therewith), said method comprising administering a subject in need thereof (for example a subject suffering from or predisposed to, erythromelalgia) ketamine in the form of a formulation described herein.

One or more of the formulations described herein may also be for use in the treatment, prevention, control and/or management of Epidermolysis Bullosa Epidermolysis bullosa (EB) (a group of rare inherited disorders characterized by blister formation and scarring as a result of minor mechanical trauma). Major types of EB include epidermolysis bullosa simplex, hemidesmosomal epidermolysis bullosa, junctional epidermolysis bullosa, and recessive (dystrophic) epidermolysis bullosa (RDEB).

The various formulations described herein may each be used in the treatment and/or prevention (or control) of suicidal ideation and behaviour associated with EB.

A further aspect provides a method of treating EB (or suicidal ideation and/or behaviour associated therewith), said method comprising administering a subject in need thereof (for example a subject suffering from or predisposed to, EB) ketamine in the form of a formulation described herein.

One or more of the formulations described herein may also be for use in the treatment, prevention, control and/or management of Brachioradial pruritus (a neuropathic itching condition that is characterized by unilateral or bilateral upper extremity itching).

The various formulations described herein may each be used in the treatment and/or prevention (or control) of suicidal ideation and behaviour associated with Brachioradial pruritus.

A further aspect provides a method of treating Brachioradial pruritus (or suicidal ideation and/or behaviour associated therewith), said method comprising administering a subject in need thereof (for example a subject suffering from or predisposed to Brachioradial pruritus) ketamine in the form of a formulation described herein.

Successful treatment of any of the above conditions (that is the successful treatment, prevention, control or management of any one of the conditions or disorders stated above) may be gauged by changes in the level, activity and/or expression of one or more biomarkers. Biomarkers which may be used to determine a level of treatment, prevention, management and/or control may include:

Psychological/Cognitive biomarkers—levels of anhedonia pre-treatment and post-treatment, apathy, impaired attention, motor deficits, reward-centre impairment.

Levels of certain cell types; for example, natural killer, leukocyte, neutrophil and other immune and inflammatory cell levels.

Pathways markers including, protein, steroid, pathway-dopamine levels, Mu and delta opioid, NMDA, AMPAR, sigma, receptor pathway regulation changes, immunological markers and intra-cellular communication Interleukins, cytokines, Tumour necrosis factor(s) and inflammatory pathway marker levels

One of skill will appreciate that there are assays and tests that may be used to determine levels of pain. For example, neuropathic pain may be assessed using verbal rating scales, visual analogue scales and graphic rating scales, numerical rating scale, picture or Face scales, descriptor differential scale of pain intensity (DDSI), Behavioural Measurements, Leeds Assessment of Neuropathic Symptoms and Signs (LANSS): Neuropathic Pain Questionnaire (NPQ), Quantitative sensory testing (QST) Somatosensory-evoked potentials (SEP) Standardized Evaluation of Pain (StEP): Brief Pain Inventory, Clinical Global Impression, McGill Pain Questionnaire, Palliative Care Outcome Scale.

A level of depression may be assessed using a depression rating scale such as the Hamilton Depression Rating Scale Montgomery-Asberg Depression Rating Scale, Raskin Depression Rating Scale, Beck Depression Inventory, self-report inventory Geriatric Depression Scale (GDS)e Zung Self-Rating Depression Scale i Patient Health Questionnaire (PHQ) Patient Health Questionnaire-9 (PHQ-9) Primary Care Evaluation of Mental Disorders (PRIME-MD) Patient Health Questionnaire. The Beck Depression Inventory; The Clinically Useful Depression Outcome Scale, The Inventory of Depressive Symptomatology, The Mood and Feelings Questionnaire, The Quick Inventory of Depressive Symptoms.

In terms of suicide, suicidal ideation and behaviour may be assessed using the suicidal ideation and behaviour assessment tool (SIBAT).

The formulations provided by this disclosure may be used alone or together (for example concurrently with or before and/or after) other medications. For example, any of the formulations described herein may be used together with (alongside) opioids to manage otherwise intractable pain; alongside psychotherapy to manage mood, mental health and psychiatric disorders; alongside addictive substances to support withdrawal and/or alongside medication for diseases/disorders and management of pain thereof.

One or more of the formulations described herein may also be for use in the treatment of “treatment-resistant depression”.

Treatment-resistant depression (TRD) may be defined as an episode of depression (or a condition or disease associated therewith) for which one or more treatment interventions has failed—particularly where the treatment intervention(s) have (or has) failed despite where the intervention is used for a sufficient duration of time and at adequate dose. By way of example, TRD may be defined as (i) failure to respond to one or more (drug or non-drug) anti-depressant therapy; or (ii) failure to responded to two or more currently available antidepressants of adequate dose and duration in the current episode of depression.

The various formulations described herein may each be used in the treatment and/or prevention (or control) of suicidal ideation and behaviour associated with TRD.

A further aspect provides a method of treating TRD (or suicidal ideation and/or behaviour associated therewith), said method comprising administering a subject in need thereof (for example a subject with TRD or with a history of TRD) ketamine in the form of a formulation described herein.

In a further aspect, the present invention provides a method of making any one of the formulations described herein, said method comprising combining ketamine hydrochloride (in an appropriate and/or therapeutically effective amount) together with one or more excipients, carriers or diluents, selected from the group consisting of:
(i) PEG 1500;
(ii) PEG6000;
(iii) Gelucire 44/14;
(iv) Polysorbate 80;
(v) Gelucire 48/16;
(vi) Kelcogel CGHA; and
(vii) Butylated hydroxytoluene (BHT)

A formulation prepared according to a method described herein may use or exploit an amount of one or more of the abovementioned active agent, excipients, carrier or diluent. In terms of amounts, the method of manufacture may utilise any of the amounts or quantities defined as for the first aspect of the invention.

### DETAILED DESCRIPTION

The disclosure will now be described in detail with reference to the following figures which show:

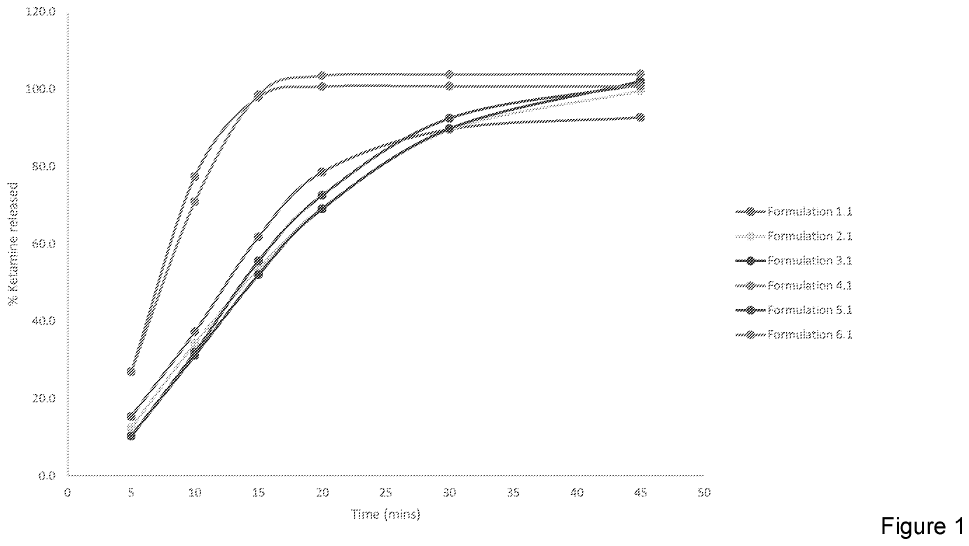

FIG. 1: Graph showing the dissolution profiles of iteration 1 formulations.

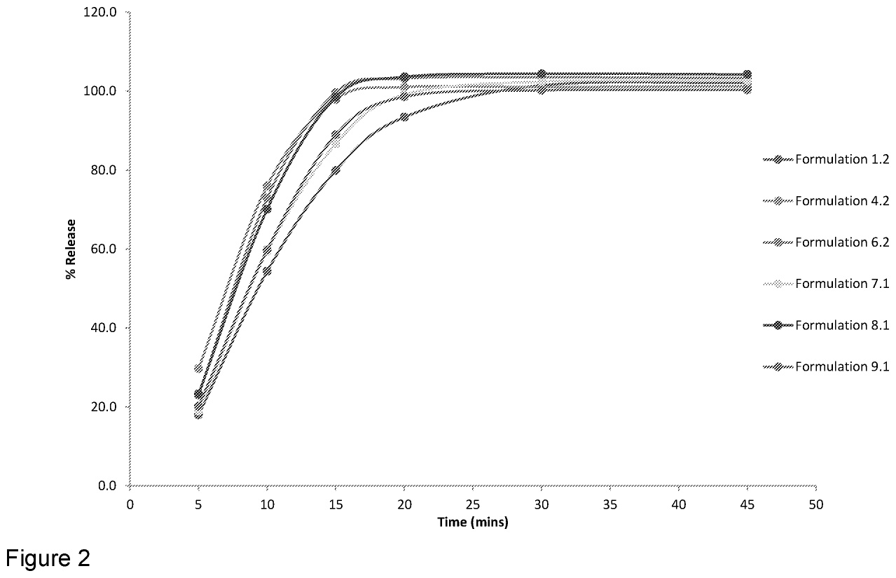

FIG. 2: Graph showing the dissolution profiles of iteration 2 formulations.

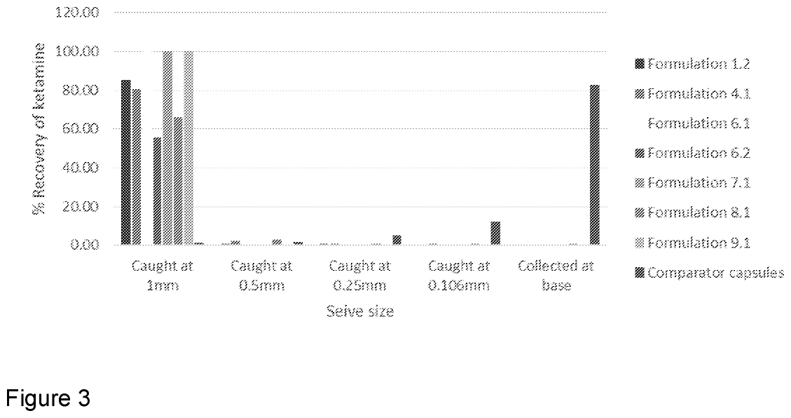

FIG. 3: A graph showing the initial insufflation results for additional testing formulations.

### EXAMPLE 1

Preparation of Filled Capsules

The composition of a number of exemplar formulations is detailed in Table 1 and Table 2.

Gelucire 44/14 was preheated in the laboratory oven at 60-70° C. and dispensed when still molten. All excipients, with the exception of Kelcogel CGHA and BHT, were weighed directly into labelled glass jars with the weights recorded in the laboratory notebook. BHT was first weighed into a weighing boat and was then added to the glass jar with the other excipients. The formulations were then placed in the laboratory oven at 60-70° C., in order to maintain the mixtures as molten. Ketamine hydrochloride was added directly into the molten formulations and stirred immediately using a micro spatula until fully wetted. Finally, Kelcogel was then added to the mixture slowly, whilst stirring.

Each sample was high shear mixed. The samples were high sheared for periods of up to three minutes, until the API appeared fully dispersed in the excipient and the formulation was visually homogeneous. The temperature of the formulation was recorded before and after high shear mixing, along with the duration of mixing. Subsequent to high shear mixing, the formulations were again retained in the oven at 60-70° C.

The formulations were each filled into white, size 1 gelatin Conisnap capsules by hand. The capsules were filled to a 500 mg±7.5% fill weight. Gelatin banding solution was prepared by weighing gelatin and water into a stainless steel pot. The solution was stirred by hand and retained in the oven at 60-70° C., until fully homogenous and ready for use. The filled capsules were banded before leaving to dry overnight. The banded capsules were then vacuum tested, using the vacuum chamber and visually inspected for any signs of leaking or defects.

Abuse Deterrence: Syringe-Ability

Using a mortar and pestle, some of the abuse deterrent properties of the formulations were assessed through syringe-ability testing. A single banded capsule was weighed and placed into the mortar. The pestle was used to break the capsule shell. The samples were ground using the pestle for two minutes, with even pressure applied for the duration of grinding. The largest fragments of shell were removed and care was taken to ensure as much formulation as possible was detached from any capsule shell fragments. 5 mL of solvent was then added to the mortar and all formulations were tested with sterile water and then 40% ethanol solution (one capsule per solvent).

Using a 23 gauge needle, 3 mL syringes were used to try extract as much material as possible from the ground formulations. The volume of sample taken up into the syringe barrel was measured. If necessary, a single syringe barrel was filled multiple times from one sample. The ejection of the material from the syringe was measured by weight, directly into a volumetric flask. The material collected in the volumetric flask was stored for analysis by assay, to determine the ketamine content.

Dissolution Testing

Six capsules from each formulation were dissolved in acidic media. Pots were filled with 250 mL of 0.01M HCl and equilibrated to 37° C.±0.5° C. using a thermometer. Single pre-weighed capsules are placed in the sample holders, consisting of a plastic mesh screen at the bottom of a glass reciprocating cylinder. The sample holder itself was then screwed into place at the relevant position on the apparatus. Aliquots of 1 mL were taken at: 5, 10, 15, 20, 30 and 45 minute time points for HPLC assay.

Insufflation Testing

Additional insufflation testing was performed in an attempt to further discriminate abuse deterrence performance between the designed prototypes. Before insufflation testing, an initial assessment was made as to whether thermal pre-treatment of the capsules was necessary. Using a single filled capsule for each formulation, shells were removed using a scalpel; the capsule fill was then immediately ground with a coffee grinder for five minutes. If for any given formulation the product was milled to a size less than 1 mm, then no thermal pre-treatment was considered necessary during further testing. If this wasn't the case, then thermal pre-treatment was used in subsequent analyses. Where thermal pre-treatment was required, capsules were frozen in a freezer at −20° C. for at least 24 hours before testing.

Shells were removed from five capsules using a scalpel, then ground with a coffee grinder for one minute. Particle size distributions of the five capsule contents were determined by pouring them onto the following sieve assembly: 1000, 500, 250, and 106 microns. Attempts could be made to further reduce any large particles by squeezing them manually by hand. The sieve was mechanically shaken for five minutes to allow formulation of the appropriate particle size to pass through. Any material that had passed through each sieve was weighed. The material on each sieve was assayed and using the approximate total capsule weight and API recovery was calculated.

Results and Discussion

Preparation of Filled Capsules

The dispensed weights for Iteration 1 and Iteration 2 formulations are detailed in 3. All capsules were filled by hand to a target fill weight of 500 mg±7.5%. Capsules were banded, dried overnight and vacuum tested at <−25″ Hg for twenty minutes. A visual assessment of the capsules was made and no capsules were found to be leaking or contained any defects.

TABLE 3
Dispensing Weights from formulations from Iteration 1
Dispensing weights (g)

| | Iteration 1 - Formulations | | | | | |
|---|---|---|---|---|---|---|
| | 1.1 | 2.1 | 3.1 | 4.1 | 5.1 | 6.1 |
| Material | 1268/101/01 | 1268/101/02 | 1268/101/03 | 1268/101/04 | 1268/101/05 | 1268/101/06 |
| Kelcogel CGHA | 6.4276 | 5.5229 | 6.4374 | 6.4360 | 6.4353 | 6.4370 |
| PEG1500 | — | — | — | 9.1956 | 1.8485 | 5.5160 |
| PEG6000 | — | 0.9229 | 1.8374 | — | — | — |
| Gelucire 44/14 | 11.9552 | 11.0360 | 10.1081 | — | — | 4.1475 |
| Gelucire 48/16 | — | — | — | — | 10.1285 | — |
| Polysorbate 80 | — | 0.9317 | — | 2.7524 | — | 2.3177 |
| BHT | 0.0205 | 0.0201 | 0.0203 | 0.0198 | 0.0200 | 0.0202 |
| Ketamine HCI | 1.6023 | 1.6020 | 1.6014 | 1.6002 | 1.6025 | 1.6003 |

Abuse Deterrence: Syringe-Ability

“Syringe-ability” testing (as one measure of abuse deterrence) was performed using 5 mL of solvent, a 23 gauge needle and a 3 mL syringe barrel. Tests were completed using both ethanol and sterile water. The results presented in 4 show the weight of the ejected material and the corresponding amount of ketamine recovered from assay.

For tests conducted in sterile water, an amount of formulation 5.1 was able to be drawn up into the syringe barrel and ejected through the needle. For tests conducted using 40% ethanol, some material was able to be drawn up and ejected from the syringe for all formulations. Most API was recovered from formulation 2.1 and formulation 3.1, with formulation 1.1 and formulation 4.1 having the lowest values of ejection.

TABLE 4
Syringe-ability Results for Formulations in 40% Ethanol and sterile water

| | | Iteration 1 - Formulations | | | | | |
|---|---|---|---|---|---|---|---|
| Solvent | Ejection weights | 1.1 | 2.1 | 3.1 | 4.1 | 5.1 | 6.1 |
| Ethanol | Total | 0.4524 | 1.0623 | 1.3226 | 0.3532 | 0.7462 | 0.9143 |
| 40% | Ejection (g) | | | | | | |
| | Ketamine | 4.0 | 7.9 | 8.3 | 3.2 | 6.4 | 7.7 |
| | Recovered (mg) | | | | | | |
| Sterile | Total | 0.0000 | 0.0000 | 0.0000 | 0.0000 | 0.0390 | 0.0000 |
| Water | Ejection (g) | | | | | | |
| | Ketamine | 0.0 | 0.0 | 0.0 | 0.0 | 0.3 | 0.0 |
| | Recovered (mg) | | | | | | |

Dissolution Testing

Capsules for each formulation from Iteration 1 underwent dissolution screening. The results from the HPLC analysis of the dissolution samples are detailed in Table 5. The capsules were analysed in sets of six for each of the formulations, with the average of the six results reported. All six formulations reached the criteria for immediate release, with 80% recovery at 45 minutes. The slowest releases were observed in formulation 2.1 and formulation 3.1 with an 80% recovery being measured at 30 minutes. Formulation 4.1 and formulation 6.1 gave the fastest dissolution profiles with an 80% release occurring within 15 minutes time point.

TABLE 5
Dissolution results for Iteration 1 formulations

| | % ketamine hydrochloride recovered | | | | | |
|---|---|---|---|---|---|---|
| Time | Formulation | Formulation | Formulation | Formulation | Formulation | Formulation |
| Point | 1.1 | 2.1 | 3.1 | 4.1 | 5.1 | 6.1 |
| 5 mins | 15.3 | 12.4 | 10.3 | 26.9 | 10.2 | 27.0 |
| 10 mins | 37.3 | 34.3 | 31.2 | 70.9 | 32.0 | 77.4 |
| 15 mins | 61.8 | 53.6 | 52.0 | 98.5 | 55.6 | 97.9 |
| 20 mins | 78.6 | 69.3 | 69.0 | 103.5 | 72.5 | 100.7 |
| 30 mins | 89.6 | 89.4 | 89.8 | 103.8 | 92.5 | 100.8 |
| 45 mins | 92.6 | 99.6 | 102.1 | 104.0 | 101.2 | 100.7 |

Discussion

From the syringe-ability results obtained, formulation 1.1 and formulation 4.1 showed the greatest potential for abuse deterrence formulations. The dissolution data generated showed that formulation 4.1 and formulation 6.1 provided the fastest release of API and scope for increasing abuse deterrence properties, whilst maintaining immediate release dissolution performance. For these formulations the inclusion of either or both PEG1500 and Gelucire 44/14, at comparatively high levels, may be attributable to the favourable syringe-ability and/or dissolution results. Formulations that had higher levels of Gelucire 44/14 tended to have slower release profiles but performed well for abuse deterrence screening. Formulations comprising PEG1500 provided desirable dissolution release times; it was noted that the abuse deterrent properties varied.

Formulation 1.1, formulation 4.1 and formulation 6.1 were progressed, with minor adjustments made to their compositions, to improve either or both the dissolution profile and abuse deterrence properties. Three new formulations were designed to determine the effect of varying levels of PEG1500, Gelucire 44/14 and Polysorbate 80 on, for example, abuse deterrent properties and dissolution. The aim was to identify formulations that exhibit acceptable abuse deterrence but retain a usable drug dissolution profile.

### EXAMPLE 2 (MODIFICATIONS OF FORMULATIONS 1.1, 4.1 AND 6.1)

Preparation of Filled Capsules

The dispensed weights for the 2^{nd} iteration formulations are detailed in Table 6. All capsules were filled by hand to 500 mg±7.5%. Gelatin banding solution, #1268/114/03, was prepared. Capsules were banded, dried overnight and vacuum tested. A visual assessment of the capsules was made and no capsules were found to be leaking or contained any defects.

TABLE 6
Dispensing weights for Iteration 2 formulations
Dispensing weights (g)

| | Iteration 2 - Formulations | | | | | |
|---|---|---|---|---|---|---|
| | 1.2 | 7.1 | 8.1 | 4.2 | 9.1 | 6.2 |
| Material | 1268/113/01 | 1268/113/02 | 1268/113/03 | 1268/113/04 | 1268/114/01 | 1268/114/02 |
| Kelcogel CGHA | 11.0332 | 11.0319 | 9.6528 | 9.6566 | 9.6534 | 9.6514 |
| PEG1500 | — | — | 8.9864 | 11.0340 | 2.7561 | 8.9659 |
| PEG6000 | — | — | — | — | — | — |
| Gelucire 44/14 | 16.5337 | 14.4724 | 8.9912 | 3.1024 | 15.1823 | 6.9028 |
| Polysorbate 80 | — | 2.0725 | — | 3.7977 | — | 2.0749 |
| BHT | 0.0302 | 0.0303 | 0.0304 | 0.0297 | 0.0302 | 0.0297 |
| Ketamine HCl | 2.4033 | 2.4152 | 2.4328 | 2.4069 | 2.4015 | 2.3869 |

Abuse Deterrence: Syringe-Ability

The abuse deterrence syringe-ability testing was performed using 5 mL of solvent, a 23 gauge needle and a 3 mL syringe barrel. Tests were completed using both ethanol and sterile water. The results presented in Table 7 show the weight of the ejected material and the corresponding measured ketamine recovered from assay. No material was taken up in the syringe using water as the solvent for any of the formulations and as a consequence, only results using 40% ethanol as the solvent were used to discriminate between the formulations. Formulation 1.2 and formulation 7.1 gave the best results in this round of testing, although the differences between the formulations was not as great as seen with the formulations prepared for Example 1.

Although there is some variation between the performance of the formulations tested, they all demonstrated desirable injection deterrence—particularly as no extraction in water occurred (it is assumed that this would be the likely solvent for preparation for injection by an abuser).

TABLE 7
Syringe-ability results for Iteration 2 formulations

| | | Iteration 2 - Formulations | | | | | |
|---|---|---|---|---|---|---|---|
| Solvent | Ejection weights | 1.2 | 7.1 | 8.1 | 4.2 | 9.1 | 6.2 |
| Ethanol | Average Total | 2.6019 | 2.2941 | 3.3928 | 3.7483 | 3.2572 | 3.0925 |
| 40% | Ejection (g) | | | | | | |
| | Ketamine | 19.9 | 17.7 | 28.8 | 31.7 | 27.2 | 26.0 |
| | Recovered (mg) | | | | | | |
| | Rel. Std. Dev | 16.4 | 10.1 | 9.2 | 4.7 | 1.3 | 0.6 |
| Sterile | Total | 0.0000 | 0.0000 | 0.0000 | 0.0000 | 0.0000 | 0.0000 |
| Water | Ejection (g) | | | | | | |
| | Ketamine | 0.0 | 0.0 | 0.0 | 0.0 | 0.0 | 0.0 |
| | Recovered (mg) | | | | | | |

Dissolution Testing

The results from the HPLC analysis of the dissolution samples are detailed in Table 8. The capsules were analysed in sets of six for each of the formulations, with the average of the six results reported. All six formulations reached the criteria for immediate release, with 80% recovery at 45 minutes. The fastest releases were observed in formulation 4.2, formulation 6.2 and formulation 8.1, with an 80% recovery being measured by 15 minutes. Formulation 1.2 had the slowest release profile. It was noted that the difference between the fastest and the slowest formulations are much smaller than for iteration 1. All six profiles were considered to be meet the criteria for an immediate release product.

TABLE 8
Dissolution results for Iteration 2 formulations

| | % ketamine hydrochloride recovered | | | | | |
|---|---|---|---|---|---|---|
| Time | Formulation | Formulation | Formulation | Formulation | Formulation | Formulation |
| Point | 1.2 | 4.2 | 6.2 | 7.1 | 8.1 | 9.1 |
| 5 mins | 18.0 | 29.7 | 23.0 | 18.9 | 23.3 | 20.2 |
| 10 mins | 54.4 | 76.0 | 72.9 | 59.4 | 70.1 | 59.8 |
| 15 mins | 79.9 | 97.9 | 99.7 | 86.7 | 98.5 | 88.9 |
| 20 mins | 93.4 | 101.0 | 103.2 | 99.1 | 103.6 | 98.6 |
| 30 mins | 101.5 | 101.2 | 103.5 | 102.4 | 104.4 | 100.2 |
| 45 mins | 102.1 | 101.2 | 103.3 | 102.6 | 104.3 | 100.3 |

Discussion (Example 2)

The syringe-ability results suggested that all the formulations display abuse deterrent properties, with formulations containing greater levels of Gelucire 44/14 generally performing better. All formulations were able to resist any material being drawn up in the syringe when water was used as the solvent. This supports the abuse deterrence nature of the formulations. The dissolution profiles of the new formulations are largely improved from those tested in Example 1 and all six formulations released above 80% by the twenty minute time point. Therefore all the formulations satisfy an immediate release profile.

The results from both Examples 1 and Examples 2 present several useful formulations. The positive syringe-ability data gave confidence of an abuse deterrence classification for most of the formulations assessed. The dissolution profiles for the majority of the formulations are exemplar for immediate release products and the data gathered so far suggests high replicability, with low relative standard deviations.

Seven of the best formulations were selected as lead formulations and were prepared again. The capsules were prepared for further syringe-ability testing in order to reinforce the abuse deterrence data already gathered. A higher number of capsules were tested to reduce any discrepancies in the results. In addition to further syringe-ability testing, the capsules were subjected to an initial insufflation test, ahead of a full abuse deterrence screening work stage.

### EXAMPLE 3

Additional Syringe Ability and Insufflation Testing

Preparation of Filled Capsules

The dispensed weights for capsules prepared for additional testing are detailed in Table 9. All capsules were filled by hand to 500 mg±7.5%. Gelatin banding solutions, #1268/128/01 and #1268/129/01, were prepared. Capsules were banded using the Qualiseal bench banding machine, dried overnight and vacuum tested at <−25″ Hg for twenty minutes. A visual assessment of the capsules was made and no capsules were found to be leaking or contained any defects.

TABLE 9
Dispensing weights for capsules for additional testing
Dispensing weights (g)

| | Additional Testing | | | | | | |
|---|---|---|---|---|---|---|---|
| | 1.2 | 4.1 | 6.1 | 6.2 | 7.1 | 8.1 | 9.1 |
| Material | 1268/122/01 | 1268/122/02 | 1268/123/01 | 1268/123/02 | 1268/124/01 | 1268/124/02 | 1268/124/03 |
| Kelcogel CGHA | 11.0754 | 48.4200 | 9.6462 | 9.6823 | 11.0261 | 9.6550 | 9.6443 |
| PEG1500 | — | 68.9664 | 8.2725 | 8.9808 | — | 8.9830 | 2.7644 |
| Gelucire 44/14 | 16.5447 | — | 6.2000 | 6.8800 | 14.4642 | 8.9602 | 15.1585 |
| Polysorbate 80 | — | 20.6500 | 3.4500 | 2.0700 | 2.0796 | — | — |
| BHT | 0.0331 | 0.1500 | 0.0320 | 0.0306 | 0.0333 | 0.0300 | 0.0300 |
| Ketamine HCl | 2.4021 | 12.0021 | 2.4027 | 2.4018 | 2.4040 | 2.4028 | 2.4054 |

Abuse Deterrence Syringe-Ability

The abuse deterrence syringe-ability testing was performed using 5 mL of solvent, a 23 gauge needle and a 3 mL syringe barrel. Tests were completed on three capsules per formulation, using only ethanol as the formulations had been previously tested using sterile water with no formulation drawn up into the syringe. The results presented in Table 10 show the weight of the ejected material and the corresponding measured ketamine recovered from assay.

TABLE 10
Syringe-ability results for additional testing formulations

| | | Additional Testing - Formulations | | | | | | |
|---|---|---|---|---|---|---|---|---|
| Solvent | Ejection weights | 1.2 | 4.1 | 6.1 | 6.2 | 7.1 | 8.1 | 9.1 |
| Ethanol | Average Total | 2.6289 | 3.0214 | 3.3181 | 3.2811 | 3.0034 | 3.1166 | 2.9613 |
| 40% | Ejection (g) | | | | | | | |
| | Ketamine | 20.48 | 22.12 | 26.12 | 26.52 | 25.16 | 26.12 | 22.68 |
| | Recovered (mg) | | | | | | | |
| | Rel. Std. Dev. | 19.4 | 5.9 | 12.0 | 5.0 | 7.2 | 7.5 | 7.8 |

Initial Insufflation Testing

Capsules from each of the additional testing formulations, as well as powder-filled ketamine capsules, were assessed for insufflation. The results from the testing are presented in Table 11. In comparison to the powder ketamine capsules, all six of the abuse deterrent formulations showed reduced ability to move through sieves with decreasing mesh sizes. Three of the formulations (Formulation 6.1, Formulation 7.1 and Formulation 9.1) were unable to be sieved through the largest mesh size and had the full contents of the ground formulation collected at the first sieve. As these formulations did not pass through any of the sieves, the recovered API contents were not measured so a theoretical 100.00% API recovery is applied. For formulations that had some ground particles pass through some of the sieves, only very low amounts of ketamine were recovered. The smallest sieve used had a mesh size of 0.106 mm, which roughly corresponds to particle sizes that are able to be passed through the nasal mucosa, conducting insufflation. None of the ground abuse deterrent formulations were able to pass through this size of sieve and demonstrate limitation to insufflation. Above 80.00% of the powder-filled capsules was passed through all of the sieves and collected at the sieve base. The results suggest that the powder filled ketamine capsules are susceptible to insufflation, with the majority of particles being below the smallest mesh size. In relation to the abuse deterrent formulations, the powder filled capsules performed poorly.

The total API recovered for each of the formulations, with the exception of those that did not sieve through any mesh, represent the amount collected and analysed in relation to the total maximum ketamine possible. The process of insufflation testing, inevitably results in the loss of some material in transference. This explains why the total for each formulation is not always 100.00%. The method for grinding and transferring the formulations aims to replicate the methods that an abuser may apply.

TABLE 11
Initial insufflation results for additional testing formulations

| | Amount of total API recovered % | | | | | | | |
|---|---|---|---|---|---|---|---|---|
| | | | | | | | | Powder-filled |
| Sieve | | | | | | | | ketamine |
| size | 1.2 | 4.1 | 6.1 | 6.2 | 7.1 | 8.1 | 9.1 | capsules |
| 1 mm | 85.30 | 80.23 | 100.00* | 55.60 | 100.00* | 65.85 | 100.00* | 1.02 |
| 0.5 mm | 0.02 | 2.06 | 0.00 | 0.09 | 0.00 | 2.66 | 0.00 | 1.61 |
| 0.25 mm | 0.00 | 0.34 | — | 0.00 | — | 0.17 | — | 4.80 |
| 0.106 mm | — | 0.01 | — | — | — | 0.02 | — | 11.84 |
| Base | — | 0.00 | — | — | — | 0.00 | — | 82.67 |

*Theoretical recovery - no formulation was passed through the largest sieve mesh so no analysis was conducted on the formulation

### SUMMARY

The syringe-ability analysis performed on the formulations suggested that each formulation had abuse deterrent properties, with reduced recovery of ketamine for each dosage. Formulation 1.2 and Formulation 4.1 yielded the lowest API recovery % for the seven tested formulations. Insufflation analysis was used to further discriminate between the formulations.

Insufflation testing suggested that all the formulations provided good abuse deterrent properties, with either no or very little formulation ground to small particle sizes. This provides some verification of the abuse deterrent nature of these formulations. Although all of the formulations produced encouraging results during testing, the three formulations that had no sieved particles showed the greatest potential for formulations to resist grinding into smaller particles.

### CONCLUSION

Abuse deterrent ketamine formulations were designed and prepared. In Example 1, a number of formulations showed promising syringe-ability and dissolution results. The results suggested that the PEG1500 and Gelucire 44/14 content may have contributed to favourable syringe-ability and/or dissolution results. Formulations that had higher levels of Gelucire 44/14 tended to have slower release profiles, but performed well for abuse deterrence screening. Formulations comprising PEG1500 provided desirable dissolution release times, but differing levels in the formulation compositions impacted the abuse deterrent properties of the formulations.

In Example 2, formulation 1.1, formulation 4.1 and formulation 6.1 were progressed, with minor adjustments made to their compositions to generate three new formulations. In general, improvements in the dissolution profile were shown and all six formulations released above 80% by the twenty minute time point. All formulations demonstrated desirable injection deterrence as no extraction in water occurred (this being the likely solvent for preparation for injection by an abuser).

The results from both Example 1 and Example 2 presented several useful formulations.

1. A composition comprising:
(i) an active agent comprising ketamine and/or pharmaceutically acceptable salts, enantiomers, derivatives or metabolites thereof;
(ii) one or more carrier compounds/compositions;
(iii) a viscosity modifier; and
(iv) optionally, an antioxidant.

2. The composition of claim 1, formulated for oral administration

3. The composition of claim 1, wherein the ketamine and/or pharmaceutically acceptable salts, enantiomers, derivatives or metabolites thereof is selected from the group consisting of:
(i) ketamine hydrochloride;
(ii) racemic (R,S) ketamine hydrochloride;
(iii) R-ketamine;
(iv) S-Ketamine;
(v) 2R,6R-Hydroxynorketamine hydrochloride;
(vi) 2S,6S-Hydroxynorketamine hydrochloride;
(vii) cis-6-Hydroxynorketamine hydrochloride;
(viii) (S)-(+)-Ketamine hydrochloride;
(ix) Norketamine hydrochloride;
(x) (R)-Norketamine hydrochloride; and
(xi) (S)-Norketamine hydrochloride.

4. The composition of claim 1, wherein the composition comprises 2%, 3%, 4%, 5%, 6%, 7%, 8%, 9%, 10%, 15%, 20%, 25%, 30%, 35%, 40%, 45% (v/v %) ketamine hydrochloride.

5. The composition of claim 1, wherein the one or more carrier compound(s) is selected from the group consisting of:
(i) co-polymers;
(ii) triglycerides;
(iii) poloxamers;
(iv) polysorbates;
(v) polyethoxylates;
(vi) polyethylene glycols;
(vii) poly-glycerides;
(viii) poloxamer 124;
(ix) polysorbate 80;
(x) polysorbate 20;
(xi) kolliphor EL;
(xii) Kolliphor RH40;
(xiii) Polyethylene glycol 1000;
(xiv) Polyethylene glycol 1500;
(xv) Polyethylene glycol 2000;
(xvi) Polyethylene glycol 6000;
(xvii) Polyethylene glycol 8000;
(xviii) Kolliphor HS15;
(xix) Gelucire 44/14;
(xx) Gelucire 48/16; and
(xxi) Gelucire 50/13.

6. The composition of claim 1, wherein the one or more carrier compounds are selected from the group consisting of:
(i) 1% and 50% (v/v %) PEG 1500;
(ii) 7%, 8%, 9%, 10%, 15%, 20%, 25%, 26%, 27%, 30%, 40%, 41%, 42%, 43%, 44%, 45%, 46%, 47%, 48% or 49% (v/v %) PEG 1500;
(iii) 9.19% (v/v %) PEG 1500;
(iv) 27.57% (v/v %) PEG 1500; and
(v) 45.95% (v/v %) PEG 1500.

7. The composition of claim 1, wherein the one or more carrier compounds are selected from the group consisting of:
(i) 1% and 20% (v/v %) PEG 6000;
(ii) 2%, 3%, 4%, 5%, 6%, 7%, 8%, 9%, 10%, 12%, 15%, 17% or 19% (v/v %) PEG 6000;
(iii) 4.6% (v/v %) PEG 6000; and
(iv) 9.19% (v/v %) PEG 6000.

8. The composition of claim 1, wherein the one or more carrier compound(s) comprises or further comprises a carrier compound selected from the group consisting of:
(i) Gelucire 44/14;
(ii) Polysorbate 80; and/or
(iii) Gelucire 48/16.

9. The composition of claim 1, wherein the carrier compound comprises Gelucire 44/14 at between 5% and 70% (v/v %).

10. The composition of claim 1, wherein the carrier compound comprises 20.68% (v/v %) Gelucire 44/14, 50.55% (v/v %) Gelucire 44/14, 55.14% (v/v %) Gelucire 44/14 or 59.74% (v/v %) Gelucire 44/14.

11. The composition of claim 1, wherein the carrier compound comprises Polysorbate 80 at between 1% and 30% (v/v %)

12. The composition of claim 11, wherein the carrier compound comprises 4.6% (v/v %) Polysorbate 80; 11.49% (v/v %) Polysorbate 80 or 13.79% (v/v %) Polysorbate 80.

13. The composition of claim 1, wherein the carrier compound comprises Gelucire 48/16 at between 10% and 70% (v/v %).

14. The composition of claim 13, wherein the carrier compound comprises 50.55% (v/v %) Gelucire 48/16.

15. The composition of claim 1, wherein the viscosity modifier is selected from the group consisting of
(i) a gum;
(ii) a gellan gum having two acyl substituents-acetate and glycerates;
(iii) a gum derived from Sphingomonas elodea;
(iv) a high acyl gellan gum having two acyl substituents;
(v) a gum comprising a repeating unit of the tetrasaccharide polymer which consists of two residues of D-glucose and one of each residues of L-rhamnose and D-glucuronic acid and
(vi) Kelcogel® CGHA™ or Kelcogel® LT100.

16. The composition of claim 1, wherein the viscosity modifier comprises Kelcogel CGHA at between 20% and 50% (v/v %).

17. The composition of claim 16, wherein the viscosity modifier comprises 27.57% (v/v %) or 32.17% (v/v %) Kelcogel CGHA.

18. The composition of claim 1, wherein the antioxidant is Butylated hydroxytoluene (BHT).

19. A composition comprising ketamine hydrochloride and one or more excipients, carriers or diluents, selected from the group consisting of:
(i) PEG1500;
(ii) PEG6000;
(iii) Gelucire 44/14;
(iv) Polysorbate 80;
(v) Gelucire 48/16;
(vi) Kelcogel CGHA; and
(vii) Butylated hydroxytoluene (BHT).

20. The composition of claim 19, comprising ketamine hydrochloride; Kelcogel CGHA; Gelucire 44/14; and Butylated hydroxytoluene (BHT).

21. The composition of claim 19, comprising ketamine hydrochloride; Kelcogel CGHA; PEG 6000; Gelucire 44/14; Polysorbate 80; and Butylated hydroxytoluene (BHT).

22. The composition of claim 19, comprising comprise ketamine hydrochloride; Kelcogel CGHA; PEG 6000; Gelucire 44/14; and Butylated hydroxytoluene (BHT).

23. The composition of claim 19, comprising ketamine hydrochloride; Kelcogel CGHA; PEG1500; Polysorbate 80; and Butylated hydroxytoluene (BHT).

24. The composition of claim 19 comprising ketamine hydrochloride; Kelcogel CGHA; PEG1500; Gelucire 48/16; and Butylated hydroxytoluene (BHT).

25. The composition of claim 19 comprising ketamine hydrochloride; Kelcogel CGHA; PEG1500; Gelucire 44/14; Polysorbate 80; and Butylated hydroxytoluene (BHT)

26. The composition of claim 19 comprising ketamine hydrochloride; Kelcogel CGHA; Gelucire 44/14 and Butylated hydroxytoluene (BHT)

27. The composition of claim 19 comprising ketamine hydrochloride; Kelcogel CGHA; Gelucire 44/14; Polysorbate 80 and Butylated hydroxytoluene (BHT)

28. The composition of claim 19 comprising ketamine hydrochloride; Kelcogel CGHA; PEG1500; Gelucire 44/14 and Butylated hydroxytoluene (BHT).

29. The composition of claim 19 comprising ketamine hydrochloride; Kelcogel CGHA; PEG1500; Gelucire 44/14; Polysorbate 80 and Butylated hydroxytoluene (BHT)

30. The composition of claim 19 comprising ketamine hydrochloride; Kelcogel CGHA; PEG1500; Gelucire 44/14 and Butylated hydroxytoluene (BHT)

31. The composition of claim 19 comprising ketamine hydrochloride; Kelcogel CGHA; PEG1500; Gelucire 44/14; Polysorbate 80 and Butylated hydroxytoluene (BHT).

32. A method of managing, treating and/or preventing pain or suicidal ideation and behaviour associated with pain, neurological disorders, mood disorders and mental health disorders, said method comprising administering a subject a composition according to claim 1.

33. A method of managing, treating and/or preventing pain or suicidal ideation and behaviour associated with pain, neurological disorders, mood disorders and mental health disorders, said method comprising administering a subject a composition according to claim 19.

34.-41. (canceled)

42. A composition of claim 1, wherein the composition is encapsulated.

43. A composition of claim 19, wherein the composition is encapsulated in a hard capsule.

44. The composition of claim 1, wherein the composition is resistant to misuse and/or abuse.

45. The composition of claim 19, wherein the composition is resistant to misuse and/or abuse.

46. The composition of claim 1, wherein the composition exhibits the immediate release of the active agent.

47. The composition of claim 19, wherein the composition exhibits the immediate release of the active agent.

48. The composition of claim 1, wherein dissolution of the active agent from the composition equates to an 80% recovery of the active agent after 30-45 minutes.

49. The composition of claim 19, wherein dissolution of the active agent from the composition equates to an 80% recovery of the active agent after 30-45 minutes.

50. The composition of claim 1, wherein the composition comprises 1%-50% (v/v %) ketamine and/or pharmaceutically acceptable salts, enantiomers, derivatives or metabolites thereof.
